(19) United States
(12) Patent Application Publication
(10) Pub. No.: US 2025/0064878 A1
(43) Pub. Date: Feb. 27, 2025

(54) TRADITIONAL CHINESE MEDICINE COMPOSITION FOR EYEBROW ENHANCEMENT, EYEBROW GROWTH POWDER, PREPARATION METHOD AND USE THEREOF

(71) Applicant: Guangdong Pharmaceutical University, Guangzhou City (CN)

(72) Inventors: Mingzhu XIAO, Guangzhou City (CN); Letao Bo, Guangzhou City (CN); Bohan Zhang, Guangzhou City (CN)

(21) Appl. No.: 18/813,266

(22) Filed: Aug. 23, 2024

(30) Foreign Application Priority Data
Aug. 23, 2023 (CN) 202311064949.3

(51) Int. Cl. A61K 36/236; A61K 9/16; A61K 36/14; A61K 36/185; A61K 36/28; A61K 36/605; A61K 36/638; A61K 36/64; A61K 36/744; A61P 17/14

(52) U.S. Cl. CPC A61K 36/236; A61K 9/1641; A61K 36/14; A61K 36/185; A61K 36/28; A61K 36/605; A61K 36/638; A61K 36/64; A61K 36/744; A61P 17/14

(57) ABSTRACT

The disclosure provides a traditional Chinese medicine composition for eyebrow enhancement, an eyebrow growth powder, a preparation method and use thereof, and relates to the technical field of traditional Chinese medicines. The traditional Chinese medicine composition for eyebrow enhancement comprises the following components in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus and 3-5 parts of Ligustri Lucidi Fructus.

### CROSS REFERENCE TO THE RELATED APPLICATIONS

This application is based upon and claims priority to Chinese Patent Application No. 202311064949.3, filed on Aug. 23, 2023, the entire contents of which are incorporated herein by reference.

### TECHNICAL FIELD

The disclosure relates to the technical field of traditional Chinese medicines, in particular to a traditional Chinese medicine composition for eyebrow enhancement, eyebrow growth powder, a preparation method and use thereof.

### BACKGROUND

The ancients said, “The most enchanting sight is that fleeting smile, with myriad charms lingering around the eyebrows.”. As one of the five senses, eyebrows are naturally very important. Good eyebrows can often increase people's charm and make people look round in a charming manner. Eyebrows play a decisive role in people's image. Eyebrows have a protective effect on the eyes, and they are located at the upper part of the orbit, thus blocking the sweat and dust falling from the forehead for the eyes. However, some people are troubled by the phenomenon that they are born with few eyebrows, or their hair falls off due to acquired factors. In order to make up for the scarce eyebrow pencil, methods such as embroidery with needles and knives are often used, but the eyebrows obtained by such methods are not as natural as those obtained by natural eyebrows.

At present, there are many products on the market that use chemically synthesized drugs as effective ingredients to promote eyebrow growth, but basically all of them have the shortcomings of long onset period, irritation to the skin, and certain side effects after long-term use, so it is difficult to achieve the ideal effect.

### SUMMARY OF THE DISCLOSURE

In order to solve the above problems, the disclosure provides a traditional Chinese medicine composition for eyebrow enhancement, eyebrow growth powder, a preparation method and use thereof. The disclosure takes traditional Chinese medicines as raw materials, has no toxic chemical components, and is based on the theory of traditional Chinese medicine, so as to prescribe the right medicine, which can achieve the effects of preventing eyebrow loss and promoting eyebrow growth, and has the advantages of rapid onset, stable drug effect and high safety.

In order to achieve the above objectives, the present disclosure provides the following technical solutions:

The present disclosure provides a traditional Chinese medicine composition for eyebrow enhancement, wherein comprising the following components in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus and 3-5 parts of Ligustri Lucidi Fructus.

The present disclosure also provides an eyebrow growth powder, wherein comprising the following raw materials in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus, 3-5parts of Ligustri Lucidi Fructus, 35 parts of glycerol tristearate, 60 parts of soybean lecithin, 45 parts of poloxamer 188, and 5 parts of water.

The present disclosure also provides a method for preparing the eyebrow growth powder according to the above technical scheme, wherein comprising:
1) pulverizing and mixing the chuanxiong rhizome, mulberry, sesame, Eclipta Herba, Platycladi cacumen, Rehmanniae Radix Preparata, Gardeniae Fructus and Ligustri Lucidi Fructus to obtain a compound;
2) mixing the compound obtained in step 1) with the glycerol tristearate to obtain an oil phase;
3) mixing the soybean lecithin, poloxamer 188 and water to obtain a water phase;
4) mixing the oil phase obtained in step 2) with the water phase obtained in step 3) to obtain a mixed phase, and sequentially carrying out high-speed shearing, ultrasonic treatment and high-pressure homogenization on the mixed phase to obtain a mixture;
5) freeze-drying the mixture obtained in step 4) to obtain the eyebrow growth powder.

In some embodiments, a particle size of the compound in step 1) is less than 100 meshes.

In some embodiments, a mixing temperature in step 2) is 80° C., and a mixing is carried out in a water bath.

In some embodiments, a mixing temperature in step 3) is 100° C., and a mixing is carried out in a water bath.

In some embodiments, the conditions of high-speed shearing in step 4) comprise: a rotating speed is 1000 rpm, and a time is 1-2 min;
the conditions of ultrasonic treatment comprise: a time is 2 min, a power is 1000 w, and a frequency is 50 kHz;
the conditions of high pressure homogenization comprise: a time is 4-6 min and a pressure is 50000 psi.

In some embodiments, the conditions of freeze-drying in step 5) comprise: pre-freezing the mixture at −10° C. for 4-6 h, and then freezing at −45° C. and 1 kPa for 24-36 h.

The present disclosure also provides a use of the traditional Chinese medicine composition or the eyebrow growth powder according to the above technical scheme in preparing a medicine for preventing eyebrow shedding.

The present disclosure also provides a use of the traditional Chinese medicine composition or the eyebrow growth powder according to the above technical scheme in preparing an eyebrow growth medicine.

In the disclosure, the source, efficacy and compatibility principle of the raw materials of the eyebrow growth powder are as follows:

Chuanxiong rhizome, a dry rhizome of chuanxiong rhizome in Umbelliferae, is pungent and warm, and belongs to liver, gallbladder and heart meridian. It has the effects of promoting blood circulation, removing blood stasis, promoting qi circulation and depression, dispelling pathogenic wind and relieving pain. According to the disclosure, the pharmacological characteristics of chuanxiong rhizome for promoting blood circulation and qi circulation and descending blood sea are utilized to cool blood, promote blood circulation and promote hair growth; in addition, the disclosure also uses coumarin components in chuanxiong rhizome to generate reactive oxygen species under ultraviolet irradiation, thereby protecting hair follicles, activating hair follicle cells, promoting hair growth, and promoting blood circulation and hair growth.

Mulberry, a mature fruit of mulberry, a deciduous tree of Moraceae, is sweet, sour and cold in nature, and has the functions of promoting fluid production, nourishing yin and nourishing blood and moistening intestines. The disclosure can effectively treat the problems of blood deficiency and yin deficiency, dry and sparse hair, shedding and the like by utilizing the pharmacological characteristics of mulberry returning to kidney meridian and nourishing yin. In addition, mulberry can make the body fluid abundant by promoting fluid production and moistening dryness, which is helpful to improve hair vitality.

Sesame, the seed of Sesamum indicum, is flat in nature and sweet in taste. It enters the liver and kidney meridians, and has the effects of nourishing liver and kidney, nourishing essence and blood, and moistening intestines. The disclosure uses the pharmacological characteristics of sesame to tonify liver and kidney and nourish essence and blood, and is used for premature gray hair, alopecia after illness and alopecia due to liver and kidney deficiency. In addition, sesame is rich in nutrients, which is beneficial to hair growth and increase melanin, and has the functions of dandruff removal, anti-drying and deep regulation.

Platycladi cacumen, which belong to the dry branches and leaves of thuja in Cupressaceae, are cold in nature and bitter in taste. They belong to lung, liver and spleen meridians, and have the effects of cooling blood, stopping bleeding, resolving phlegm and relieving cough. According to the disclosure, the bitter and cold leaves of Platycladi cacumen are used for cooling blood, expelling wind and promoting hair growth and blackening hair by taking advantage of the pharmacological characteristics of entering blood, cooling blood to stop bleeding and entering liver meridian, which are equivalent to those of Polygonum multiflorum Thunb, Ligustri Lucidi Fructus and Eclipta Herba, and have a therapeutic effect on liver and kidney deficiency and essence and blood deficiency. In addition, the disclosure also utilizes flavonoids in the leaves of Platycladi cacumen to better exert the antibacterial and anti-inflammatory effects of the product.

Eclipta Herba, a dry aerial part of the yerbadetajo herb of a herb, is sweet, sour and cold, and enters the liver and kidney meridians. In the disclosure, the characteristics that Eclipta Herba is sour and sweet, turns into yin, and enters the liver and kidney meridians are utilized to nourish the liver and kidney, so as to promote the growth of black beard of the composition.

Ligustri Lucidi Fructus, belonging to Oleaceae, is flat in nature, sweet and bitter in taste, and belongs to liver and kidney meridians. It has the effects of nourishing yin, prolonging life, strengthening liver and kidney, clearing heat and improving eyesight. In the disclosure, Ligustri Lucidi Fructus and Eclipta Herba should be used together, which is helpful for treating premature gray hair caused by liver and kidney yin deficiency.

Rehmanniae Radix Preparata, the rhizome of Scrophulariaceae plant rehmannia or huaiqing rhizoma rehmanniae, is bitter and cold in nature, and has the effects of nourishing blood, nourishing yin, replenishing essence and filling marrow. In the disclosure, the symptoms of hair loss, hair whitening and the like caused by deficiency of qi and blood can be improved by utilizing the characteristics of the prepared rhizome of rehmannia to enrich blood and nourish yin and enter the liver and kidney channels.

Gardeniae Fructus, the fruit of Gardenia jasminoides of Rubiaceae, is bitter and cold in nature, and enters the heart, lungs and triple energizer meridians. It has the effects of purging fire, removing annoyance, clearing away heat and promoting diuresis, cooling blood and detoxicating, and relieving swelling and pain for external use. In the present disclosure, Gardeniae Fructus is used to treat hematemesis and hematochezia caused by excessive fire in triple energizer. In addition to blood, reduce blood loss to raise hair.

The traditional Chinese medicine composition provided by the disclosure is based on the theory of traditional Chinese medicine, and the symptomatic single medicines are compatible, so that the efficacy and safety are greatly improved.

In the disclosure, the compatibility principle of the traditional Chinese medicine composition is shown in FIG. 1:

Eclipta Herba is the monarch drug, Platycladi cacumen and Ligustri Lucidi Fructus are the ministers, black sesame, Rehmanniae Radix Preparata and mulberry are the adjuvant drugs, and Chuanxiong rhizome and Gardenia Fructus are the emissaries. On the right side, dark gray bottom is the main symptom, white bottom is the etiology and pathogenesis, and light gray bottom is the secondary symptom.

“Wang Zhen Zun Jing” says that “if eyebrows are moist, full of blood; if dry, and weak in blood.” Deficiency of qi and blood leads to empty hair orifices and loss of nutrition. According to the “Xiaoer Weisheng Zongwei Lunfang”, “eyebrows belong to the liver, and the if the qi of liver is not glorious, so eyebrows are not born for a long time.” Also the kidney stores essence, the liver stores blood, and the essence and blood of liver and kidney are homologous, which breed each other and provide nutrition for eyebrow growth, so the scarcity of eyebrows is mostly caused by deficiency of liver and kidney. Fang Zhong Eclipta Herba, is sweet and cold, enters the liver and kidney meridians. It can tonify kidney essence, nourish liver blood, nourish hair orifices, and clear blood heat when it tastes cold, so it is a monarch drug. Adjuvant drug adopts Ligustri Lucidi Fructus to nourish the yin of liver and kidney, blacken the hair, and brighten the eyes and ears, and it is compatible with Eclipta Herba to cultivate yin and blood deficiency; Platycladi cacumen enter the liver meridian to help Eclipta Herba cool blood and dispel wind, and have the effect of hair growth and blackening hair. Black sesame, Rehmanniae Radix Preparata, mulberry and Ligustri Lucidi Fructus, Eclipta Herba can nourish liver and kidney, nourish yin and nourish blood, “kidney is the innate foundation”, kidney essence is full, blood vitality is active, hair is nourished and hair grows, as the saying goes, “kidney is full, teeth are firm and eyebrows are long”; Chuanxiong rhizome, Gardenia Fructus and Platycladi cacumen can cool blood, dispel wind, clear away heat, promote blood circulation and remove stagnation, and treat wind, heat, deficiency and blood stasis caused by disharmony of viscera and qi and blood. All the medicines are used together, which complement each other, and play the roles of benefiting essence and nourishing blood, promoting blood circulation and removing blood stasis, cooling blood and expelling wind, so that the hair can be raised and there is no danger of falling off.

The disclosure has the beneficial effects that:

Compared with the existing products using chemical substances as effective substances for eyebrow growth, this product adopts traditional Chinese herbal medicines as raw materials, which has no irritation to the skin, and the stability and safety of Chinese herbal medicines are higher. The product has the advantages of quick effect and lasting effect. The disclosure studies the mechanism of eyebrow shedding and eyebrow growth through the theory of traditional Chinese medicine, and uses Chinese herbal medicines to suit the remedy to the case, so that the eyebrow growth effect is quick and obvious.

### BRIEF DESCRIPTION OF THE DRAWINGS

In order to more clearly explain the technical scheme in the embodiment of the present disclosure or in the prior art, the drawings needed in the embodiment will be briefly introduced below.

FIG. 1 shows the compatibility principle of the traditional Chinese medicine composition;

FIG. 2 is an experimental grouping diagram;

FIG. 3 is a comparison chart of the first day of each group's medication in example 1 of the present disclosure;

FIG. 4 is a comparison chart of the eighth day of each group's medication in example 1 of the present disclosure;

FIG. 5 is a comparison chart of the 10th day of each group's medication in example 1 of the present disclosure;

FIG. 6 is a comparison chart of the 12th day of each group's medication in example 1 of the present disclosure;

FIG. 7 is a comparison chart of the 15th day of each group's medication in example 1 of the present disclosure.

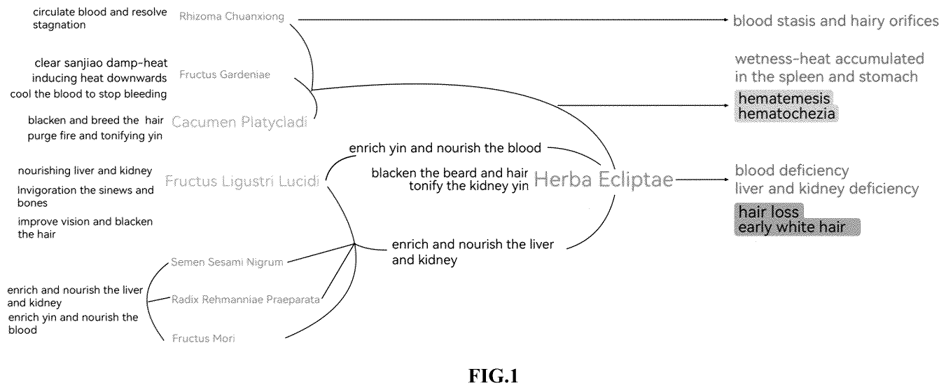

### DETAILED DESCRIPTION OF THE EMBODIMENTS

The present disclosure provides a traditional Chinese medicine composition for eyebrow enhancement, which comprises the following components in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus and 3-5 parts of Ligustri Lucidi Fructus.

The present disclosure also provides an eyebrow growth powder, wherein comprising the following raw materials in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus, 3-5 parts of Ligustri Lucidi Fructus, 35 parts of glycerol tristearate, 60 parts of soybean lecithin, 45 parts of poloxamer 188, and 5 parts of water.

The present disclosure also provides a method for preparing the eyebrow growth powder according to the above technical scheme, wherein comprising:
1) pulverizing and mixing the chuanxiong rhizome, mulberry, sesame, Eclipta Herba, Platycladi cacumen, Rehmanniae Radix Preparata, Gardeniae Fructus and Ligustri Lucidi Fructus to obtain a compound;
2) mixing the compound obtained in step 1) with the glycerol tristearate to obtain an oil phase;
3) mixing the soybean lecithin, poloxamer 188 and water to obtain a water phase;
4) mixing the oil phase obtained in step 2) with the water phase obtained in step 3) to obtain a mixed phase, and sequentially carrying out high-speed shearing, ultrasonic treatment and high-pressure homogenization on the mixed phase to obtain a mixture;
5) freeze-drying the mixture obtained in step 4) to obtain the eyebrow growth powder.

In the disclosure, chuanxiong rhizome, mulberry, sesame, Eclipta Herba, Platycladi cacumen, Rehmanniae Radix Preparata, Gardeniae Fructus and Ligustri Lucidi Fructus are crushed and mixed to obtain a compound. In the present disclosure, the traditional Chinese medicine is preferably washed, drained and dried, and then subjected to superfine grinding, and the conditions for superfine grinding are not particularly limited in the present disclosure, and those skilled in the art can follow the routine. In the present disclosure, the particle size of the compound is preferably less than 100 meshes.

In the disclosure, the obtained compound is mixed with glycerol tristearate to obtain an oil phase. In the present disclosure, the mixing temperature is preferably 80° C., and the mixing is carried out in a water bath.

In the disclosure, the soybean lecithin, poloxamer 188 and water are mixed to obtain a water phase. In the present disclosure, the mixing temperature is preferably 100° C., and the mixing is carried out in a water bath.

In the disclosure, the obtained oil phase is mixed with the obtained water phase to obtain a mixed phase, and the mixed phase is sequentially subjected to high-speed shearing, ultrasonic treatment and high-pressure homogenization to obtain a mixture. In the present disclosure, the conditions of high-speed shearing preferably include: the rotation speed is 1000 rpm, and the time is 1-2 min. In the present disclosure, the conditions of the ultrasonic treatment preferably include: the time is 2 min, the power is 1000 w, and the frequency is 50 kHz. In the present disclosure, the conditions of high-pressure homogenization preferably include: the time is 4-6 min, and the pressure is 50000 psi.

In the disclosure, the obtained mixture is freeze-dried to obtain eyebrow growth powder. In the present disclosure, the conditions of freeze-drying process preferably include: pre-freezing the mixture at −10° C. for 4-6 hours, and then freezing at −45° C. and 1 kPa for 24-36 hours.

The disclosure also provides the use of the traditional Chinese medicine composition or the eyebrow growth powder in preparing a medicine for preventing eyebrow shedding. The disclosure has no special limitation on the dosage form and preparation method of the medicine, and the skilled in the art can adopt routine methods.

The disclosure also provides the use of the traditional Chinese medicine composition or the eyebrow growth powder in preparing an eyebrow growth medicine. The disclosure has no special limitation on the dosage form and preparation method of the medicine, and the skilled in the art can adopt routine methods.

In the present disclosure, the application method of the eyebrow growth powder preferably includes: taking 0.3 g of the eyebrow growth powder, adding 10 ml of boiling water, uniformly mixing until the powder is suspended, and cooling to room temperature, that is, the eyebrow growing liquid medicine is obtained, and dipping the eyebrow growing liquid medicine with a cotton swab every day after cleansing, and smearing 2-5 ml along the direction of eyebrow growth.

In order to further illustrate the present disclosure, the following examples will be described in detail, but they cannot be understood as limiting the scope of protection of the present disclosure.

### Example 1

This example provides a traditional Chinese medicine composition powder for eyebrow enhancement, specifically as follows:

5 parts of chuanxiong rhizome, 8 parts of mulberry, 5 parts of sesame, 20 parts of Eclipta Herba, 10 parts of Platycladi cacumen, 5 parts of Rehmanniae Radix Preparata, 3 parts of Gardeniae Fructus and 3 parts of Ligustri Lucidi Fructus.

The preparation method of the traditional Chinese medicine composition comprises the following:

S1, chuanxiong rhizome, mulberry, sesame, Eclipta Herba, Platycladi cacumen, Rehmanniae Radix Preparata, Gardeniae Fructus and Ligustri Lucidi Fructus were weighed according to the parts by weight, then washed, drained, dried, After ultra-fine grinding for 60 s, it was sieved through 100 mesh to obtain a mixed powder.

S2, 35 parts by weight of glycerol tristearate was added into the mixed powder, and heated in a water bath at 80° C. to obtain an oil phase.

S3, 60 parts by weight of soybean lecithin and 45 parts by weight of poloxamer 188 were weighed, and 5 parts by volume of distilled water was added, stirred and dissolved, and heated in a water bath at 100° C. to obtain a water phase.

S4, the oil phase prepared by S2 and the water phase prepared by S3 were mixed, and sheared at high speed for 1.5 min at a rotating speed of 1000 rpm; then ultrasonic treated for 2 min, with the power of 1000 w and frequency of 50 kHz; then homogenized under high pressure for 5 min at a pressure of 50000 psi to obtain a mixed solution.

S5, the mixed solution was put in a rotary evaporator, pre-freezed at −10° C. for 5 h, and the freeze-drying temperature was adjusted to −45° C., the pressure was adjusted to 1 kPa, and the time was adjusted to 30 h, so as to prepare a Chinese medicine freeze-dried powder, namely the eyebrow growth powder.

### Example 2

This example provides a Chinese medicine composition powder for eyebrow growth. Compared with Example 1, the only difference is as follows:

2 parts of chuanxiong rhizome, 5 parts of mulberry, 3 parts of sesame, 10 parts of Eclipta Herba, 20 parts of Platycladi cacumen, 5 parts of Rehmanniae Radix Preparata, 5 parts of Gardeniae Fructus and 3 parts of Ligustri Lucidi Fructus.

### Example 3

This example provides a Chinese medicine composition powder for eyebrow growth. Compared with Example 1, the only difference is as follows:

5 parts of chuanxiong rhizome, 8 parts of mulberry, 3 parts of sesame, 15 parts of Eclipta Herba, 15 parts of Platycladi cacumen, 3 parts of Rehmanniae Radix Preparata, 3 parts of Gardeniae Fructus and 5 parts of Ligustri Lucidi Fructus.

### Example 4

### (A) Induction of Growth Period

In this experiment, the traditional Chinese medicine composition was used as the experimental drug, and cold boiled water was used as the blank control to carry out the hair growth experiment of mice. The specific experimental operation is as follows.
(1) The mice were divided into groups and treated with drugs.

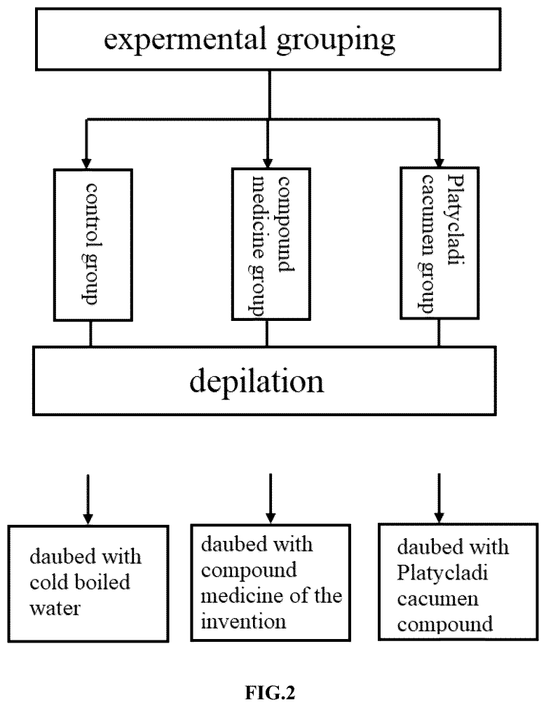

C57BL/6J mice purchased by Guangdong Zhiyuan Biomedical Technology Co., Ltd. were used as experimental animals. At first, Weiting depilatory cream (from France, produced by Reckitt Benckiser (China) Co., Ltd.) was used to shave the back hair of each mouse, so that the skin on the back was bare and pink. Then the experimental mice were divided into three groups (blank control group, eyebrow powder group prepared in Example 1 and Platycladi cacumen group), with 7 mice in each group. The experimental grouping is shown in FIG. 2.

Medication: 0.3 g raw eyebrow powder was taken and added into 10 ml ultrapure water, and stirred to make it into suspension. After cooling to room temperature, the liquid medicine was dipped with a cotton swab and smeared on the back of mice. The daubing frequency of the three groups of mice was once a day, each time was about 1 ml. The whole depilation area on the back of mice was divided into three parts, namely, upper, middle and lower parts, which were administered separately, while the blank control group was daubed with the same amount of cold boiled water.
(2) Experimental result

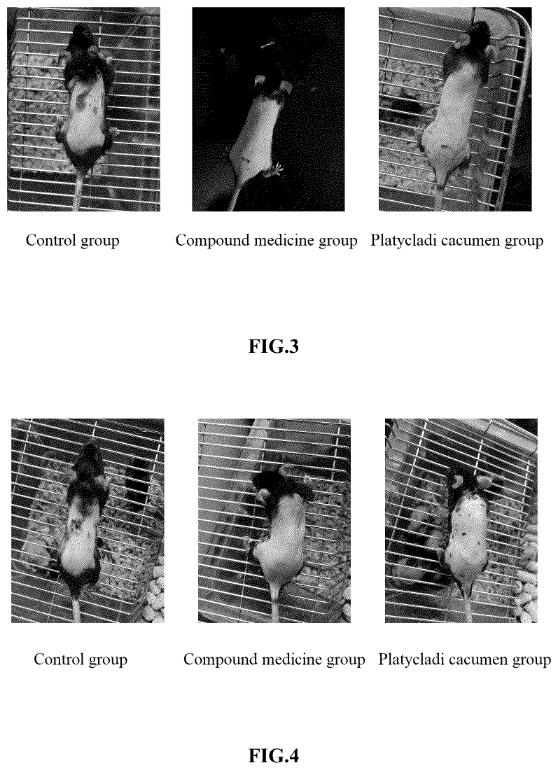

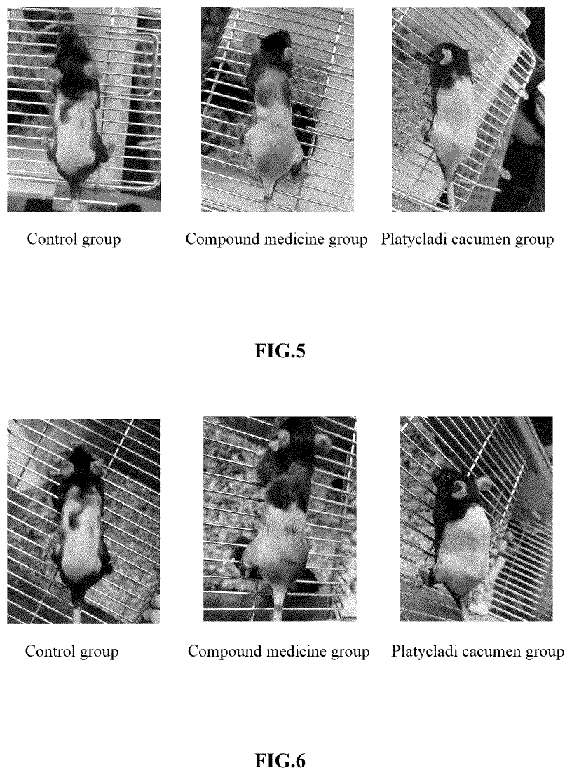

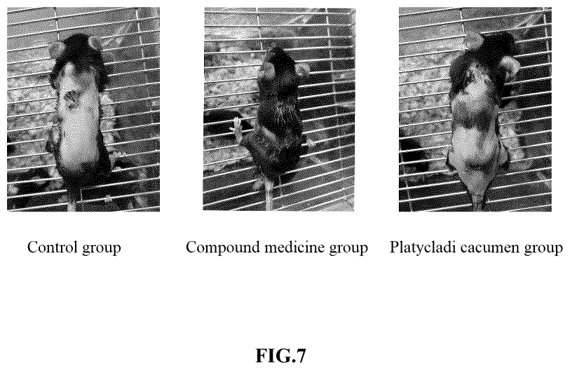

The experimental results are shown in FIGS. 3-7. From left to right, there are blank group, compound medicine group (eyebrow powder prepared in Example 1) and Platycladi cacumen group. The results on the first day of administration (Jun. 1, 2023) are shown in FIG. 3, from which it can be seen that there is no change in the three groups of mice on the first day of administration. The results of the eighth day of administration (Jun. 8, 2023) are shown in FIG. 4. As can be seen from FIG. 4, the upper part of the back of the mice in the compound medicine group of the present disclosure began to turn gray, while the other two groups had no obvious changes. The results on the 10th day of administration (Jun. 10, 2023) are shown in FIG. 5. From FIG. 5, it can be seen that the lower part of the back of the mice in the compound medicine group of the present disclosure began to turn gray, and the upper part began to turn black, while the other two groups had no obvious changes. The results on the 12th day of administration (Jun. 12, 2023) are shown in FIG. 6. From FIG. 6, it can be seen that the lower part of the back of the mice in the compound medicine group of the present disclosure is gray, and the upper part begins to grow short hair, while the back of the Platycladi cacumen group begins to turn gray, and there is no obvious change in the blank group. The results on the 15th day of administration (Jun. 15, 2023) are shown in FIG. 7. From FIG. 7, it can be seen that black hair has grown on the back of the mice in the compound medicine group of the present disclosure, the back of the Platycladi cacumen group began to turn black and the blackened area expanded, and the skin of the blank group began to turn gray.

### (B) Clinical Trials

There were 10 volunteers with sparse eyebrows. After cleaning their faces every night, 2-5 ml of eyebrow growth liquid (0.3 g eyebrow growth powder and 10 ml boiled water) was applied along the direction of eyebrow growth. The trial period was 21 days. The length, density, gloss and patient satisfaction of eyebrows before and after treatment were observed. The length, density and gloss were evaluated by 0-2 points (poor), 3-4 points (general), 5-6 points (good) and 7-8 points (very good) respectively. The patient satisfaction was evaluated by ten points. The higher the score, the higher the patient satisfaction. The results are shown in Table 1.

TABLE 1
Post-use Scoring Table

Eyebrow Growth Index

| | | | | Patient |
|---|---|---|---|---|
| Group | Length | Density | Gloss | satisfaction |
| 1 | 4 | 4 | 6 | 9 |
| 2 | 6 | 7 | 7 | 8 |
| 3 | 5 | 5 | 5 | 9 |
| 4 | 6 | 6 | 8 | 9 |
| 5 | 7 | 5 | 4 | 10 |
| 6 | 8 | 8 | 5 | 9 |
| 7 | 6 | 4 | 6 | 9 |
| 8 | 8 | 8 | 8 | 10 |
| 9 | 7 | 5 | 7 | 8 |
| 10 | 5 | 6 | 6 | 10 |

Eyebrow length: (normally it grows about 0.2-0.5 mm every day, and it grows about 1-1.5 cm a month).
0-1 points: the eyebrow growth length is about 0-1 mm;
2-4 points: the eyebrow growth length is about 2-3 mm;
5-7 points: the eyebrow growth length is about 4-8 mm;
8-10 points: the eyebrow growth length is about 8 mm or more.

### Eyebrow Density:

0-1 points: extremely sparse, with less than 10 hairs in a single visual field;
2-4 points: sparse, clearly visible skin, with less than 20 hairs in a single visual field;
5-7 points: medium density, almost invisible skin, the number of hairs in a single visual field is ≥20, <30;
8-10 points: dense, the skin is invisible, and the number of hairs in a single visual field is ≥30.

### Eyebrow Gloss:

0-1 points: dull;
2-4 points: average luster;
5-7 points: the hair is healthy and shiny;
8-10 points: the hair is black, bright and shiny.

### Patient Satisfaction:

0-3 points: the drug is irritating after using the medicine, and the hair-increasing effect is average;
4-8 points: there is no irritation after using the medicine, and the hair-increasing effect is satisfactory;
9-10 points: there is no irritation after using the medicine, and the hair-increasing effect is very satisfactory.

All the data were entered into Excel for proofreading, and SPSS statistical software was used to process the data. The counting data were described by rate, and χ² test was used for comparison between groups. The measurement data were described by x̅±s, and t test was used for comparison between groups. P<0.05 is statistically significant.

Although the above embodiment has described the invention in detail, it is only a part of the embodiment of the invention, not the whole embodiment. Skilled in the art can also obtain other embodiments according to this embodiment without inventing, and these embodiments all belong to the scope of protection of the invention.

1. A traditional Chinese medicine composition for eyebrow enhancement, wherein comprising the following components in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus and 3-5 parts of Ligustri Lucidi Fructus.

2. An eyebrow growth powder, wherein comprising the following raw materials in parts by weight: 2-5 parts of chuanxiong rhizome, 5-8 parts of mulberry, 3-5 parts of sesame, 10-20 parts of Eclipta Herba, 10-20 parts of Platycladi cacumen, 3-5 parts of Rehmanniae Radix Preparata, 3-5 parts of Gardeniae Fructus, 3-5 parts of Ligustri Lucidi Fructus, 35 parts of glycerol tristearate, 60 parts of soybean lecithin, 45 parts of poloxamer 188, and 5 parts of water.

3. A method for preparing the eyebrow growth powder according to claim 2, wherein comprising:
1) pulverizing and mixing the chuanxiong rhizome, mulberry, sesame, Eclipta Herba, Platycladi cacumen, Rehmanniae Radix Preparata, Gardeniae Fructus and Ligustri Lucidi Fructus to obtain a compound;
2) mixing the compound obtained in step 1) with the glycerol tristearate to obtain an oil phase;
3) mixing the soybean lecithin, poloxamer 188 and water to obtain a water phase;
4) mixing the oil phase obtained in step 2) with the water phase obtained in step 3) to obtain a mixed phase, and sequentially carrying out high-speed shearing, ultrasonic treatment and high-pressure homogenization on the mixed phase to obtain a mixture;
5) freeze-drying the mixture obtained in step 4) to obtain the eyebrow growth powder.

4. The method according to claim 3, wherein a particle size of the compound in step 1) is less than 100 meshes.

5. The method according to claim 3, wherein a mixing temperature in step 2) is 80° C., and a mixing is carried out in a water bath.

6. The method according to claim 3, wherein a mixing temperature in step 3) is 100° C., and a mixing is carried out in a water bath.

7. The method according to claim 3, wherein the conditions of high-speed shearing in step 4) comprise: a rotating speed is 1000 rpm, and a time is 1-2 min;
the conditions of ultrasonic treatment comprise: a time is 2 min, a power is 1000 w, and a frequency is 50 kHz;
the conditions of high pressure homogenization comprise: a time is 4-6 min and a pressure is 50000 psi.

8. The method according to claim 3, wherein the conditions of freeze-drying in step 5) comprise: pre-freezing the mixture at −10° C. for 4-6 h, and then freezing at Δ45° C. and 1 kPa for 24-36 h.

9. Use of the traditional Chinese medicine composition according to claim 1 or the eyebrow growth powder in preparing a medicine for preventing eyebrow shedding.

10. Use of the traditional Chinese medicine composition according to claim 1 or the eyebrow growth powder in preparing an eyebrow growth medicine.
